(19) United States
(12) Patent Application Publication
(10) Pub. No.: US 2007/0224689 A1
(43) Pub. Date: Sep. 27, 2007

(54) Method for predicting or identifying the onset of premature membrane rupture during pregnancy

(75) Inventors: RameshBabu Boga, Alpharetta, GA (US); James M. Takeuchi, Roswell, GA (US); J. Gavin MacDonald, Decatur, GA (US)

Correspondence Address: DORITY & MANNING, P.A., POST OFFICE BOX 1449, GREENVILLE, SC, 29602-1449, US

(73) Assignee: Kimberly-Clark Worldwide, Inc., Neenah, WI (US)

(21) Appl. No.: 11/799,097

(22) Filed: Apr. 30, 2007

Related U.S. Application Data
(63) Continuation-in-part of application No. 10/790,617, filed on Mar. 1, 2004.

(51) Int. Cl. G01N 33/50; G01N 30/00

(52) U.S. Cl. 436/86; 436/161

(57) ABSTRACT

A diagnostic test kit for simultaneously detecting the presence of amines (e.g., putrescine, cadaverine, tyramine, and trimethylamine) and C-reactive protein in a vaginal sample is provided. The test kit includes a lateral flow assay device containing a chromatographic medium (e.g., porous membrane, fluidic channel, etc.). The chromatographic medium defines a first detection zone within which is contained an amine-sensitive chromogen and a second detection zone within which is contained an immunoreactive receptive material that preferentially binds with C-reactive protein or a specific binding member thereof. The first and second detection zones may produce signals that are detectable, either visually or through the use of instrumentation. In this manner, the kit provides a complimentary system for both predicting and identifying the onset of premature membrane rupture. For example, the detection of amines in the vaginal sample may serve as a diagnosis of bacterial vaginosis, which may provide an early warning of the potential for premature membrane rupture. The complimentary detection of CRP may serve as an indicator of rupture and thus alert the patient to seek immediate medical care. Such a system may be equally effective in point of care (POC) and over-the-counter (OTC) applications.

### RELATED APPLICATIONS

The present application is a continuation-in-part of U.S. patent application Ser. No. 10/790,617, filed on Mar. 1, 2004, the contents of which are incorporated herein by reference.

### BACKGROUND OF THE INVENTION

Premature rupture of the amnichorionic membrane prior to the onset of labor is becoming an increasing problem for pregnant women. Upon premature rupture of the membrane, the fetus must be promptly delivered when the mother becomes clinically infected or the fetus shows signs of potential compromise. In either case, if left untreated, possible death to the fetus and the mother could result. The timing for the delivery of the baby becomes critical, as the risk of intrauterine infection increases significantly as more time passes following rupture. Accordingly, it becomes critical to provide a method of early detection of rupture. The problem is that leaking amniotic fluid is frequently confused by the mother with her own urine or vaginal discharge. This results in many false alarms and unnecessary trips to either the doctor's office or to the hospital for evaluation of the pregnant woman to rule out possible rupture of the membrane. The conventional test for assessing premature rupture of the membrane is for the physician to observe the cervix after employing a speculum in an effort to identify pooling of fluid behind the cervix. The physician then applies a swab of pH paper held by a forceps to the fluid located in the area of the cervix. Because amniotic fluid is more alkaline than nominal vaginal fluid, the pH paper reacts to its presence by turning purple-blue. Unfortunately, pH cannot be used as a tool for definitive diagnosis and it is effective only after rupture of the membrane. It has long been recognized in the medical profession, however, that the prevention of preterm delivery or premature rupture of fetal membranes is preferable.

In this regard, extensive research has been performed to find biochemical markers for the prediction of impending premature rupture of fetal membranes. One such biomarker that has been discovered to be present in vaginal fluid upon membrane rupture is C-reactive protein. (DiNaro et al., “C-reactive Protein in Vaginal Fluid of Patients with Preterm Premature Rupture of Membranes”, Acta Obstet. Gynecol. Scand., 82, 1072-1079 (2003)). Although a useful biomarker, C-reactive protein is not always found in vaginal fluid prior to the onset of premature membrane rupture. Thus, it is desirable to identify a biomarker that is better able to predict premature membrane rupture so that it may potentially be prevented. Unfortunately, prediction is often difficult due to the wide variety of causes of premature membrane rupture. Nevertheless, one common cause of premature membrane is bacterial vaginosis, which affects up to 40-50% of women in child-bearing age. (Hillier et al., “Association between Bacterial Vaginosis and Preterm Delivery of a Low Birth-Weight Infant” N. Engl. J. Med., 333, 1737-1742 (1995)). In bacterial vaginosis, certain amines (e.g., putrescine (1,4-diaminobutane or 1,4-DAB), cadaverine (1,5-diaminopentane or 1,5-DAP), tyramine, and trimethylamine (TMA)) are present at elevated levels in vaginal fluid. (Wolrath et al., “Trimethylamine Content in Vaginal Secretion and its Relation to Bacterial Vaginosis” APMIS, 110, 819-824 (2001 & 2002)). Therefore, the presence of high levels of putrescine, cadaverine, tyramine, and TMA in vaginal fluid is an indicative factor for bacterial vaginosis.

Thus, a need exists for a complementary diagnostic system of amines and CRP that is able to predict or identify the onset of premature membrane rupture during pregnancy.

### SUMMARY OF THE INVENTION

In accordance with one embodiment of the present invention, a method for detecting amines and C-reactive protein in a vaginal sample of a pregnant female is disclosed. The method comprises analyzing a vaginal sample of the pregnant female for the presence or absence of both amines and C-reactive protein by contacting the vaginal sample with a lateral flow device. The lateral flow device comprises a chromatographic medium that defines a first detection zone and a second detection zone, the first detection zone being capable of exhibiting a first detection signal and the second detection zone being capable of exhibiting a second detection signal. The first and second detection signal are observed. The first detection signal is correlated to the presence or absence of amines in the vaginal sample, and the second detection signal is correlated to the presence or absence of C-reactive protein in the vaginal sample.

In accordance with another embodiment of the present invention, a diagnostic test kit for detecting amines and C-reactive protein in a vaginal sample of a pregnant female is disclosed. The kit comprises detection probes conjugated with a specific binding member and a lateral flow device that contains a chromatographic medium. The medium defines a first detection zone that contains an amine-sensitive chromogen capable of producing a first detection signal. The first detection signal indicates the presence or absence of amines in the vaginal sample. The medium also defines a second detection zone that contains an immunoreactive receptive material configured to preferentially bind with the specific binding member or C-reactive protein. The detection probes are capable of producing a second detection signal when present in the second detection zone. The second detection signal indicates the presence or absence of C-reactive protein in the vaginal sample.

Other features and aspects of the present invention are discussed in greater detail below.

### BRIEF DESCRIPTION OF THE DRAWINGS

A full and enabling disclosure of the present invention, including the best mode thereof, directed to one of ordinary skill in the art, is set forth more particularly in the remainder of the specification, which makes reference to the appended figures in which:

FIG. 1 is a perspective view of one embodiment of a lateral flow assay device of the present invention;

FIG. 2 is a graphical illustration of the dose response curve generated for Example 1 in which optical density is plotted versus known C-reactive protein concentrations; and

FIG. 3 is a graphical illustration of the optical density for several of the samples of Example 2.

Repeat use of reference characters in the present specification and drawings is intended to represent same or analogous features or elements of the invention.

### DETAILED DESCRIPTION OF REPRESENTATIVE EMBODIMENTS

### Definitions

As used herein, the term “vaginal sample” generally refers to any material derived or obtained from the vagina, including vaginal fluid, amniotic fluid, etc. The material may be used as obtained or pretreated in some manner. For example, such pretreatment may include filtration, precipitation, dilution, distillation, mixing, concentration, inactivation of interfering components, the addition of reagents, lysing, etc.

### Detailed Description

Reference now will be made in detail to various embodiments of the invention, one or more examples of which are set forth below. Each example is provided by way of explanation of the invention, not limitation of the invention. In fact, it will be apparent to those skilled in the art that various modifications and variations may be made in the present invention without departing from the scope or spirit of the invention. For instance, features illustrated or described as part of one embodiment, may be used on another embodiment to yield a still further embodiment. Thus, it is intended that the present invention covers such modifications and variations as come within the scope of the appended claims and their equivalents.

Generally speaking, the present invention is directed to a method for simultaneously detecting the presence of amines (e.g., putrescine, cadaverine, tyramine, and trimethylamine) and C-reactive protein in a vaginal sample. The method employs a lateral flow assay device containing a chromatographic medium (e.g., porous membrane, fluidic channel, etc.). The chromatographic medium defines a first detection zone within which is contained an amine-sensitive chromogen and a second detection zone within which is contained an immunoreactive receptive material that preferentially binds with C-reactive protein or a specific binding member thereof. The first and second detection zones may produce signals that are detectable, either visually or through the use of instrumentation. In this manner, a complimentary system is provided for both predicting and identifying the onset of premature membrane rupture. For example, the detection of amines in the vaginal sample may serve as a diagnosis of bacterial vaginosis, which may provide an early warning of the potential for reactive premature membrane rupture. The complimentary detection of CRP may serve as an indicator of rupture and thus alert the patient to seek immediate medical care.

Such a system may be equally effective in point of care (POC) and over-the-counter (OTC) applications.

I. Amine Detection

The kit of the present invention may employ any of a variety of different amine detection mechanisms. One mechanism employs an amine-sensitive chromogen that undergoes a color change in the presence of amines (e.g., putrescine, cadaverine, tyramine, or trimethylamine) in a vaginal sample that is detectable, either visually or through instrumentation. For example, the chromogen may change from a first color to a second color, from no color to a color, or from a color to no color.

Any chromogen capable of exhibiting such a detectable change in color upon reaction with an amine may be utilized in the present invention. For example, a chromogenic reaction may be employed in which an amine reacts with a phenol reagent to produce an indophenol that has a detectable difference in color. The phenol reagent may include, for instance, phenol or phenol derivatives, such as hydroxyphenylalkyl alcohols, hydroxyphenolalkylcarboxylic acids and hydroxycinnamic acid, where the hydroxy group is in the 2 or 3-position and the alkyl group contains from 1 to 6 carbon atoms. Suitable phenol derivatives are, for example, hydroxybenzyl alcohol, hydroxyphenylacetic acid, hydroxycinnamic acid, 2-(2-hydroxyphenyl)ethanol, 2-(3-hydroxyphenyl)ethanol, 3-(2-hydroxyphenyl)propanol, 4-(2-hydroxyphenyl)butanol, 3-(2-hydroxyphenyl)propionic acid, 4-(2-hydroxyphenyl)butyric acid, 5-(2-hydyroxyphenyl)valeric acid.

The amines may directly induce a color change in the chromogen as described above. Because amines have a relatively low oxidation potential for certain phenol compounds, however, it is sometimes difficult to detect the color change (e.g., visibly). In this regard, an electron donor may optionally be employed to react with the phenol reagent and produce an intermediate compound having a higher oxidation potential for the chromogen than the amines. A variety of known electron donors may be employed for this purpose. In one embodiment, for example, halogen ions (e.g., iodide, chloride, etc.) may react with amines to form a complex having a much greater oxidation potential than the amines. Exemplary sources of ionic halogens include hydrogen iodide (HI) and water-soluble iodide salts, such as alkali metal iodide salts (e.g., potassium iodide (KI), sodium iodide (NaI), lithium iodide), ammonium iodide (NH₄I), calcium iodide (CaI₂), etc.); hydrogen chloride (HCl) and water-soluble chloride salts, such as alkali metal chloride salts (e.g., sodium hypochlorite); and so forth. If desired, an oxyhalogenation catalyst may be employed to facilitate the reaction between the electron donor and the amine. Suitable catalysts may include, for instance, salts of nitroprusside (e.g., sodium nitroprusside). If desired, the stability of such halogen-amino complexes may also be improved by maintaining the solution at an alkaline pH, such as about 9 or greater, in some embodiments about 10 or greater, and in some embodiments, about 11 or greater. A pH modifier may be employed to achieve the desired pH level. For example, the pH modifier may include an alkali metal salt, such as alkali metal hydroxides (e.g., sodium hydroxide and potassium hydroxide), alkali metal carbonates (e.g., sodium carbonate and potassium carbonate), trialkali metal phosphates (e.g., trisodium phosphate and tripotassium phosphate), and mixtures thereof.

Upon reaction with a halogen electron donor, such as described above, a halogen-amino complex is formed that has a strong oxidation potential for a phenol reagent. The halogen-amino complex may thereafter react with the phenol reagent to produce an aminophenol compound. The aminophenol compound may possess a color that is different from the original color of the phenol compound. Alternatively, the aminophenol compound may further react with excess phenol reagent to produce an indophenol that has a color that is different from the original color of the phenol compound. One example of such a reaction mechanism is known as “Berthelot's reaction” and is set forth below:

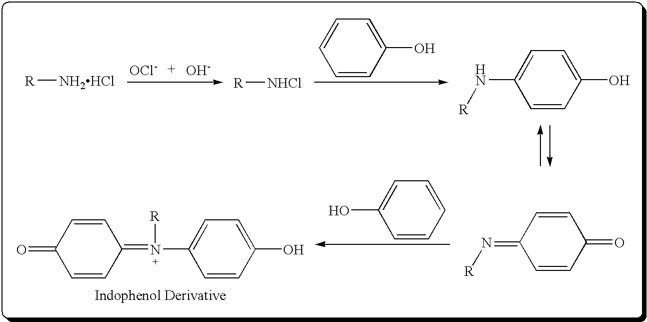

II. C-Reactive Protein (“CRP”) Detection

Any of a variety of known techniques may generally be employed in the present invention to detect the presence of C-reactive protein (“CRP”) in the vaginal sample. In one particular embodiment, for example, an immunoassay is employed that relies upon immunospecific binding reactions between CRP and a specific binding member. A particular example of a suitable immunoreactive specific binding member for CRP includes antibodies (primary or secondary). An antibody may be a monoclonal or polyclonal antibody, a recombinant protein or a mixture(s) or fragment(s) thereof, as well as a mixture of an antibody and other specific binding members. In one particular embodiment, for example, monoclonal antibodies for CRP are employed in the present invention. The details of the preparation of such antibodies and their suitability for use as specific binding members are well known to those skilled in the art. Specific binding pairs may include members that are analogs of the specific binding member. For example, a derivative or fragment of CRP, i.e., a CRP analog, may be used so long as it has at least one epitope in common with CRP.

Immunoreactive specific binding members may be employed in a variety of ways in the present invention. For example, a receptive material may be contained within a detection zone that preferentially binds with CRP or a member of a specific binding pair with CRP. Detection probes may also be employed that are conjugated with a specific binding member that preferentially binds with the receptive material or CRP. The particular selection of the specific binding member and receptive material depends on the assay format. In “sandwich” assay formats, for example, the specific binding member and receptive material are both selected to preferentially bind with CRP. In such embodiments, both the specific binding member and receptive material may be an antibody for CRP. In “competitive” assay formats, the specific binding member is selected to preferentially bind to the receptive material so that it competes with CRP for available binding sites at the receptive material. In such embodiments, the specific binding member may be CRP or an analog thereof, and the receptive material may be an antibody for CRP. Alternatively, the specific binding member may be an antibody for CRP, and the receptive material may be CRP or an analog thereof.

Any substance capable of producing a signal that is detectable visually or by an instrumental device may be used to form the detection probes. Suitable detectable substances may include, for instance, luminescent compounds (e.g., fluorescent, phosphorescent, etc.); radioactive compounds; visual compounds (e.g., colored dye or metallic substance, such as gold); liposomes or other vesicles containing signal-producing substances; enzymes and/or substrates, and so forth. Other suitable detectable substances may be described in U.S. Patent Application No. 2006/0003336 to Song, et al., which is incorporated herein in its entirety by reference thereto for all purposes. The detectable substances may be used alone or in conjunction with a particle (sometimes referred to as “beads” or “microbeads”). For instance, naturally occurring particles, such as nuclei, mycoplasma, plasmids, plastids, mammalian cells (e.g., erythrocyte ghosts), unicellular microorganisms (e.g., bacteria), polysaccharides (e.g., agarose), etc., may be used. Further, synthetic particles may also be utilized. For example, in one embodiment, latex microparticles that are labeled with a fluorescent or colored dye are utilized. Although any synthetic particle may be used in the present invention, the particles are typically formed from polystyrene, butadiene styrenes, styreneacrylic-vinyl terpolymer, polymethylmethacrylate, polyethylmethacrylate, styrene-maleic anhydride copolymer, polyvinyl acetate, polyvinylpyridine, polydivinylbenzene, polybutyleneterephthalate, acrylonitrile, vinylchloride-acrylates, and so forth, or an aldehyde, carboxyl, amino, hydroxyl, or hydrazide derivative thereof. Commercially available examples of suitable fluorescent particles include fluorescent carboxylated microspheres sold by Molecular Probes, Inc. under the trade names “FluoSphere” (Red 580/605) and “TransfluoSphere” (543/620), as well as “Texas Red” and 5- and 6-carboxytetramethylrhodamine, which are also sold by Molecular Probes, Inc. In addition, commercially available examples of suitable colored, latex microparticles include carboxylated latex beads sold by Bang's Laboratory, Inc. Metallic particles (e.g., gold particles) may also be utilized in the present invention.

When utilized, the shape of the particles may generally vary. In one particular embodiment, for instance, the particles are spherical in shape. However, it should be understood that other shapes are also contemplated by the present invention, such as plates, rods, discs, bars, tubes, irregular shapes, etc. In addition, the size of the particles may also vary. For instance, the average size (e.g., diameter) of the particles may range from about 0.1 nanometers to about 1,000 microns, in some embodiments, from about 0.1 nanometers to about 100 microns, and in some embodiments, from about 1 nanometer to about 10 microns.

The specific binding members may generally be conjugated to the probes using any of a variety of well-known techniques. For instance, covalent attachment of the specific binding members to the detection probes (e.g., particles) may be accomplished using carboxylic, amino, aldehyde, bromoacetyl, iodoacetyl, thiol, epoxy and other reactive or linking functional groups, as well as residual free radicals and radical cations, through which a protein coupling reaction may be accomplished. A surface functional group may also be incorporated as a functionalized co-monomer because the surface of the probe may contain a relatively high surface concentration of polar groups. In addition, although probes are often functionalized after synthesis, such as with poly(thiophenol), the probes may be capable of direct covalent linking with a protein without the need for further modification. For example, in one embodiment, the first step of conjugation is activation of carboxylic groups on the probe surface using carbodiimide. In the second step, the activated carboxylic acid groups are reacted with an amino group of an antibody to form an amide bond. The activation and/or antibody coupling may occur in a buffer, such as phosphate-buffered saline (PBS) (e.g., pH of 7.2) or 2-(N-morpholino) ethane sulfonic acid (MES) (e.g., pH of 5.3). The resulting probes may then be contacted with ethanolamine, for instance, to block any remaining activated sites. Overall, this process forms a conjugated probe, where the antibody is covalently attached to the probe. Besides covalent bonding, other attachment techniques, such as physical adsorption, may also be utilized in the present invention.

III. Lateral Flow Device

In accordance with the present invention, the desired reaction time between the reagents (e.g., chromogens, immunoreactive specific binding members, etc.) may be achieved by selectively controlling the medium in which the reactions occur. That is, the reaction medium is chromatographic in nature so that the reagents are allowed to flow laterally in a consistent and controllable manner. While laterally flowing through the medium, amines may react with a chromogen contained within a first discrete detection zone. Likewise, CRP may react with an immunoreactive specific binding member and/or receptive material contained within a second discrete detection zone. Due to the nature of the controlled fluid flow, any unreacted reagents travel to the end of the reaction medium so that it is unable to adversely interfere with observance of the detection zones.

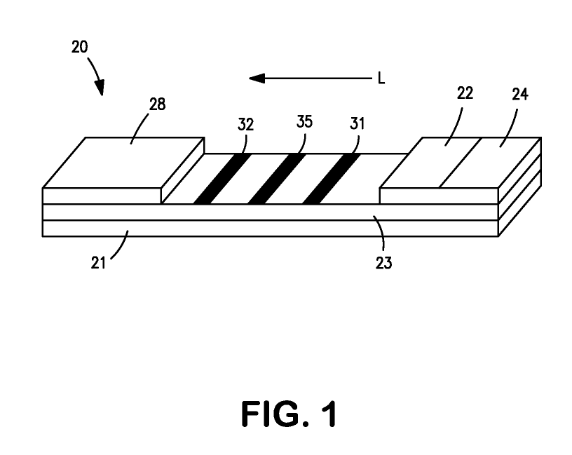

In this regard, FIG. 1 illustrates one particular embodiment of a lateral flow device 20 for simultaneously detecting the presence of amines and CRP in a vaginal sample. As shown, the lateral flow device 20 contains a chromatographic medium 23 optionally supported by a rigid support material 21. The chromatographic medium 23 may be made from any of a variety of materials through which the vaginal sample is capable of passing. For example, the chromatographic medium 23 may be a porous membrane formed from synthetic or naturally occurring materials, such as polysaccharides (e.g., cellulose materials such as paper and cellulose derivatives, such as cellulose acetate and nitrocellulose); polyether sulfone; polyethylene; nylon; polyvinylidene fluoride (PVDF); polyester; polypropylene; silica; inorganic materials, such as deactivated alumina, diatomaceous earth, MgSO₄, or other inorganic finely divided material uniformly dispersed in a porous polymer matrix, with polymers such as vinyl chloride, vinyl chloride-propylene copolymer, and vinyl chloride-vinyl acetate copolymer; cloth, both naturally occurring (e.g., cotton) and synthetic (e.g., nylon or rayon); porous gels, such as silica gel, agarose, dextran, and gelatin; polymeric films, such as polyacrylamide; and so forth. In one particular embodiment, the chromatographic medium 23 is formed from nitrocellulose and/or polyether sulfone materials. It should be understood that the term “nitrocellulose” refers to nitric acid esters of cellulose, which may be nitrocellulose alone, or a mixed ester of nitric acid and other acids, such as aliphatic carboxylic acids having from 1 to 7 carbon atoms.

The size and shape of the chromatographic medium 23 may generally vary as is readily recognized by those skilled in the art. For instance, a porous membrane strip may have a length of from about 10 to about 100 millimeters, in some embodiments from about 20 to about 80 millimeters, and in some embodiments, from about 40 to about 60 millimeters. The width of the membrane strip may also range from about 0.5 to about 20 millimeters, in some embodiments from about 1 to about 15 millimeters, and in some embodiments, from about 2 to about 10 millimeters. Likewise, the thickness of the membrane strip is generally small, such as less than about 500 micrometers, in some embodiments less than about 250 micrometers, and in some embodiments, less than about 150 micrometers.

The support 21 carries the chromatographic medium 23. For example, the support 21 may be positioned directly adjacent to the chromatographic medium 23 as shown in FIG. 1, or one or more intervening layers may be positioned between the chromatographic medium 23 and the support 21. Regardless, the support 21 may generally be formed from any material able to carry the chromatographic medium 23. The support 21 may be formed from a material that is liquid-impermeable so that fluid flowing through the medium 23 does not leak through the support 21. Examples of suitable materials for the support include, but are not limited to, glass; polymeric materials, such as polystyrene, polypropylene, polyester (e.g., Mylar® film), polybutadiene, polyvinylchloride, polyamide, polycarbonate, epoxides, methacrylates, and polymelamine; and so forth. To provide a sufficient structural backing for the chromatographic medium 23, the support 21 is generally selected to have a certain minimum thickness. Likewise, the thickness of the support 21 is typically not so large as to adversely affect its optical properties. Thus, for example, the support 21 may have a thickness that ranges from about 100 to about 5,000 micrometers, in some embodiments from about 150 to about 2,000 micrometers, and in some embodiments, from about 250 to about 1,000 micrometers. For instance, one suitable membrane strip having a thickness of about 125 micrometers may be obtained from Millipore Corp. of Bedford, Mass. under the name “SHF180UB25.”

As is well known the art, the chromatographic medium 23 may be cast onto the support 21, wherein the resulting laminate may be die-cut to the desired size and shape. Alternatively, the chromatographic medium 23 may simply be laminated to the support 21 with, for example, an adhesive. In some embodiments, a nitrocellulose or nylon porous membrane is adhered to a Mylar® film. An adhesive is used to bind the porous membrane to the Mylar® film, such as a pressure-sensitive adhesive. Laminate structures of this type are believed to be commercially available from Millipore Corp. of Bedford, Mass. Still other examples of suitable laminate device structures are described in U.S. Pat. No. 5,075,077 to Durley, III, et al., which is incorporated herein in its entirety by reference thereto for all purposes.

The device 20 may also contain an absorbent material 28 that is positioned adjacent to the medium 23. The absorbent material 28 assists in promoting capillary action and fluid flow through the medium 23. In addition, the absorbent material 28 receives fluid that has migrated through the entire chromatographic medium 23 and thus draws any unreacted components away from the detection region. Some suitable absorbent materials that may be used in the present invention include, but are not limited to, nitrocellulose, cellulosic materials, porous polyethylene pads, glass fiber filter paper, and so forth. The absorbent material may be wet or dry prior to being incorporated into the device. Pre-wetting may facilitate capillary flow for some fluids, but is not typically required. Also, as is well known in the art, the absorbent material may be treated with a surfactant to assist the wicking process.

To initiate the detection of amines and/or CRP within the vaginal sample, a user may directly apply the vaginal sample to a portion of the chromatographic medium 23 through which it may then travel in the direction illustrated by arrow “L” in FIG. 1. Alternatively, the vaginal sample may first be applied to a sample application zone 24 that is in fluid communication with the chromatographic medium 23. The sample application zone 24 may be formed on the medium 23. Alternatively, as shown in FIG. 1, the sample application zone 24 may be formed by a separate material, such as a pad. Some suitable materials that may be used to form such sample pads include, but are not limited to, nitrocellulose, cellulose, porous polyethylene pads, and glass fiber filter paper.

If desired, one or more of the reagents described above may be mixed with the vaginal sample prior to application to the device 20. For example, the vaginal sample may be allowed to mix with an electron donor (e.g., sodium hypochlorite), reaction catalyst (e.g., sodium nitroprusside), and pH modifier prior to application the device 20 so that halogen-amino complexes are formed for subsequent reaction with a phenol reagent immobilized within a detection zone. Alternatively, one or more of the reagents may be diffusively immobilized on the device 20 prior to application of the vaginal sample. This provides a variety of benefits, including the elimination of the need for a subsequent user to handle and mix the reagents with the vaginal sample or a diluent. The reagent(s) may be disposed upstream from, downstream from, or at the sample application zone 24. When disposed downstream from the point where the vaginal sample is to be applied, the vaginal sample is capable of mixing with and dissolving or re-suspending the reagents upon application. In the illustrated embodiment, for example, a reagent zone 22 is employed that is in fluid communication with the sample application zone 24. As shown in FIG. 1, the reagent zone 22 is formed from a separate material or pad. Such a reagent pad may be formed from any material through which the vaginal sample is capable of passing, such as glass fibers. Alternatively, the reagent zone 22 may simply be formed on the medium 23. Regardless, the reagent zone 22 may be applied with one or more solutions containing reagents, such as electron donors, catalysts, pH modifiers, conjugated detection probes, etc. and dried. Thus, the vaginal sample may contact the reagent zone 22 before reaching a detection zone located downstream from the reagent zone 22.

Referring again to FIG. 1, the chromatographic medium 23 also defines multiple detection zones. For instance, the chromatographic medium 23 defines a first detection zone 31 within which is contained an amine-sensitive chromogen, such as described above. For example, the first detection zone 31 may contain a phenol reagent and optionally other reagents, such as an electron donor, catalyst, pH modifier, and so forth. Regardless, amines within the vaginal sample are capable of passing through the length of the chromatographic medium 23 and reacting with the amine-sensitive chromogen within the first detection zone 31 to produce a detectable color change that may be subsequently be correlated to the presence of amines in the vaginal sample.

Any of a variety of techniques may be employed to apply the reagent(s) to the first detection zone 31. The reagent(s) may be applied directly to the chromatographic medium 23 or first formed into a solution prior to application. Various solvents may be utilized to form the solution, such as, but not limited to, water, acetonitrile, dimethylsulfoxide (DMSO), ethyl alcohol, dimethylformamide (DMF), and other polar organic solvents. The amount of the reagent(s) in the solution may range from about 0.001 to about 1 milligram per milliliter of solvent, and in some embodiments, from about 0.01 to about 0.1 milligrams per milliliter of solvent. The solution may be coated onto the chromatographic medium 23 using well-known techniques and then dried. The concentration of each reagent may be selectively controlled to provide the desired level of detection sensitivity. For example, higher concentrations may provide a higher level of detection sensitivity when low amine levels are suspected.

The reagent(s) are typically applied in such a manner that they do not substantially diffuse through the matrix of the chromatographic medium 23. This enables a user to readily detect the change in color that occurs upon reaction with an amine. For instance, the reagent(s) may form an ionic and/or covalent bond with functional groups present on the surface of the chromatographic medium 23 so that they remain immobilized thereon. In other embodiments, particles may be employed to facilitate the immobilization of the reagent(s) at the first detection zone 31. The reagent(s) may be coated or otherwise applied to particles, such as described above, which are then immobilized on the chromatographic medium 23. Although non-diffusive immobilizing techniques may be desired in some cases, it should also be understood that any other technique for applying reagent(s) to the chromatographic medium 23 may be used in the present invention. For example, certain components may be added to a reagent solution that substantially inhibit diffusion into the matrix of the chromatographic medium 23. In other cases, immobilization may not be required, and the reagent(s) may instead diffuse into the matrix of the chromatographic medium 23 for reaction with the vaginal sample.

In addition to the first detection zone 23, the chromatographic medium 23 also defines a second detection zone 35 for detecting the presence of CRP within the vaginal sample. The second detection zone 35 may be positioned downstream or upstream from the first detection zone 31. The second detection zone 35 contains an immobilized receptive material that serves as a stationary binding site for a specific binding member conjugated to a detection probe (“sandwich” assays) or for CRP (“competitive” assays). For instance, CRP has two or more binding sites (e.g., epitopes) so that upon reaching the second detection zone 35, one of these binding sites may be occupied by a first antibody conjugated to a detection probe. However, the free binding site may bind to a second antibody at the detection zone 35 to form a ternary sandwich complex. The detection probes are capable of producing a detection signal when present at the detection zone 35 that is detectable, either visually or with an instrument. Thus, the presence of the detection signal may indicate the presence or absence of CRP in the vaginal sample.

One benefit of the lateral flow device of the present invention is its ability to readily incorporate one or more additional zones to facilitate detection. For example, referring again to FIG. 1, a control zone 32 may also be employed in the lateral flow device 20 for improving detection accuracy. The control zone 32 gives a signal to the user that the test is performing properly. The control zone 32 may, for example, contain a receptive material that binds a control reagent or to reagents that do not become bound within a detection zone. These reagents may then be observed, either visually or with an instrument, within the control zone 32. The location of the control zone 32 may vary based on the nature of the test being performed. In the illustrated embodiment, for example, the control zone 32 is defined by the chromatographic medium 23 and positioned downstream from the first and second detection zones. In such embodiments, the control zone 32 may contain a material that is non-diffusively immobilized and forms a chemical and/or physical bond with probes (e.g., control probes or unbound detection probes). The detection probes may contain latex particles, for instance, that bind to a polyelectrolyte contained within the control zone 32. Various polyelectrolytic binding systems are described, for instance, in U.S. Patent App. Publication No. 2003/0124739 to Song, et al., which is incorporated herein in it entirety by reference thereto for all purposes. In alternative embodiments, however, the control zone 32 may simply be defined by a region of the absorbent material 28 to which the control reagents flow after traversing through the chromatographic medium 23. Other zones that reduce non-specific binding or non-specific adsorption of components of the vaginal sample may also be employed, such as a zone (not shown) treated with a protein, such as albumin (e.g., bovine serum albumin).

The sample application zone 24, reagent zone 22, first detection zone 31, second detection zone 35, control zone 32, and any other zone employed in the lateral flow device 20 may generally provide any number of distinct detection regions so that a user may better determine the concentration of the amines and/or CRP within the vaginal sample. Each region may contain the same or different materials. For example, the zones may include two or more distinct regions (e.g., lines, dots, etc.). The regions may be disposed in the form of lines in a direction that is substantially perpendicular to the flow of the vaginal sample through the device 20. Likewise, in some embodiments, the regions may be disposed in the form of lines in a direction that is substantially parallel to the flow of the vaginal sample through the device 20.

One particular embodiment of a method for detecting the presence of amines and CRP within a vaginal sample using the device 20 of FIG. 1 will now be described in more detail. Initially, a vaginal sample containing amines and CRP is applied to the sample application zone 24 and travels in the direction “L” to the reagent zone 22. At the reagent zone 22, sodium hypochlorite, sodium nitroprusside, and a pH modifier mix with the amines to form halogen-amino complexes. Further, colored latex particles conjugated with an antibody for CRP react with CRP to provide an intermediate binary complex. The desired reactions may occur while at the reagent zone 22 or as the mixture flows through the device 20. Regardless, the vaginal sample containing the reacted components eventually flows to the first detection zone 31, where the halogen-amino complexes react with phenol reagents. Likewise, the binary complexes bind to an antibody immobilized within the second detection zone 35 to form a ternary, sandwich complex.

After the reactions, each of the zones 31 and 35 produce a detection signal, the intensity of which may be observed visually or measured using instrumentation to qualitatively, quantitatively, or semi-quantitatively determine the level of amines and/or CRP present in the vaginal sample. For example, the intensity of the color is typically directly proportional to the concentration of amines and/or CRP. Thus, the intensity of the detection signals may be compared to a predetermined detection curve developed for a plurality of known concentrations. To determine the quantity of the amines and/or CRP in an unknown vaginal sample, the signal may simply be converted to concentration according to the detection curve.

The present invention provides a relatively simple, compact and cost-efficient kit for accurately detecting the presence of amines and CRP within a vaginal sample. The test result may be visible so that it is readily observed by the person performing the test in a prompt manner and under test conditions conducive to highly reliable and consistent test results. The test is also rapid and may be detected within a relatively short period of time. For example, the chromogen may undergo a detectable color change in less than about 30 minutes, in some embodiments less than about 10 minutes, in some embodiments less than about 5 minutes, in some embodiments less than about 3 minutes, in some embodiments less than about 1 minute, and in some embodiments, less than about 30 seconds. In this manner, the test may provide a “real-time” indication of the presence or absence of amines and/or CRP. The device may then be discarded as a unit when the test is concluded.

The present invention may be better understood with reference to the following examples.

### EXAMPLE 1

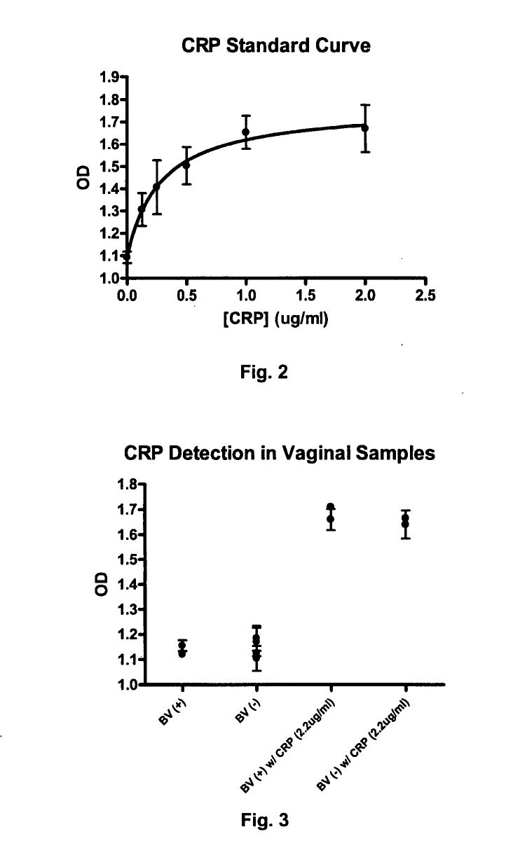

CRP monoclonal antibody (CRP MAb1 from Medimix (MedixMab), clone #6404, Lot #SP-179-2) and bovine serum albumin (BSA) buffer (10 mM phosphate buffered saline and 0.2% BSA, pH 7.3) were striped on clear-backed cards of laminated nitrocellulose (Millipore HF120). Specifically, CRP MAb1 (0.75 mg/ml) was striped at a dispense rate of 1.5 μl/cm. Two (2) BSA lines were then striped 1.25 mm on either side of the CRP MAb1 line at a dispense rate of 1.0 μl/cm. Finally, a single control line was striped downstream from the CRP MAb1 and BSA lines with 1.0 mg/ml CRP antigen (Scipac #P100-0, Lot #1049-20) at a dispense rate of 1.0 μl/cm. After striping, the cards were dried in an oven at 37° C. for 60 minutes. The cards were then assembled with an absorptive sink (Millipore CFSP203000) and a glass fiber conjugate pad (Millipore GFCP203000) pre-striped with three bands of gold particles conjugated with CRP antibody (MAb1). The conjugated particles had an optical density (“OD”) of 3.3. The assembled cards were cut into 4 mm wide strips using a guillotine cutter and then stored in plastic bags with desiccant. Assays were then performed by pipetting 2 μl of a CRP solution directly onto the nitrocellulose immediately downstream of the conjugate pad. Microtiter wells with 150 μl of TBS chase buffer (pH 7.42) were used for each test. Dose responses were generated with the six different CRP concentrations: 0.0, 0.125, 0.25, 0.5, 1.0, 2.0 μg/ml. Each strip was allowed to dry before being read in transmission mode with an optical reader. Optical density (OD) measurements from the reader were used to generate a CRP dose response standard curve. The results are set forth in FIG. 2.

### EXAMPLE 2

CRP levels were measured as described in Example 1 in vaginal samples that were known to be both positive and negative for bacterial vaginosis (“BV”) based on clinical trials conducted on non-pregnant women. Some of the vaginal samples were also spiked with CRP (2.0 μg/ml) for purposes of comparison. In addition to CRP, amine tests were also performed. More specifically, 50 μl of vaginal fluid or standards of putrescine hydrochloride solutions (5, 2.5, 1.25, 0.625, 0.312, 0.1506, 0.075, and 0.0 mg/ml) were placed in a microtiter plate wells. Thereafter, 100 μl of both a phenol-nitroprusside solution (10 mg of sodium nitroprusside and 2 ml of saturated phenol (from Sigma) in 18 ml of water) and a sodium hydroxide-hypochlorite solution (3 ml of Clorox™ bleach in 17 ml of ˜0.1N sodium hydroxide) were added to the well. The resultant solution was incubated for 10 minutes and read at 630 nanometers using microplate reader. Amine concentration of vaginal fluids was determined by a standard curve obtained with standards of putrescine hydrochloride solutions.

For purposes of comparison, the Quidel® QuikVue Advance pH and amine test was also performed. More specifically, test subjects provided swab samples of vaginal fluid. From each subject, the swab sample was tested with the commercially available QuickVue® test. A change of color to blue for both pH and amine positive was indicated as BV positive (BV+), and no change of color for either pH or amines was indicated as BV negative (BV−). The results are summarized in Table 1 and shown graphically in FIG. 3.

TABLE 1
Detection of Amines and CRP in Vaginal Samples

| | Quidel ® | Phenol-nitroprusside- | |
|---|---|---|---|
| Subject # | Test | hypochlorite Method | CRP Levels |
| 107 | BV (+) | BV (+) | Not detected |
| 117 | BV (+) | BV (+) | Not detected |
| 126 | BV (+) | BV (+) | Not detected |
| 152 | BV (+) | BV (+) | Not detected |
| 107 | — | — | 2.0 μg/ml |
| (spiked with CRP, | | | |
| 2.0 μg/ml) | | | |
| 117 | — | — | 2.0 μg/ml |
| (spiked with CRP, | | | |
| 2.0 μg/ml) | | | |
| 113 | BV (−) | BV (−) | Not detected |
| 114 | BV (−) | BV (−) | Not detected |
| 124 | BV (−) | BV (−) | Not detected |
| 130 | BV (−) | BV (−) | Not detected |
| 113 | — | — | 2.0 μg/ml |
| (spiked with CRP, | | | |
| 2.0 μg/ml) | | | |
| 114 | — | — | 2.0 μg/ml |
| (spiked with CRP, | | | |
| 2.0 μg/ml) | | | |

As indicated, none of the BV positive or negative samples had any detectable levels of CRP, while the CRP spiked samples showed levels matched by the previously generated standard curve. Further, the phenol-nitroprusside-hypochlorite amine test corresponded well with the commercially available Quidel® test.

While the invention has been described in detail with respect to the specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the appended claims and any equivalents thereto.

What is claimed is:

1. A method for identifying the onset of premature membrane rupture in a pregnant female, the method comprising:
analyzing a vaginal sample of the pregnant female for the presence or absence of both amines and C-reactive protein by contacting the vaginal sample with a lateral flow device, wherein the lateral flow device comprises a chromatographic medium that defines a first detection zone and a second detection zone, the first detection zone being capable of exhibiting a first detection signal and the second detection zone being capable of exhibiting a second detection signal;
observing the first detection signal and the second detection signal;
correlating the first detection signal to the presence or absence of amines in the vaginal sample; and
correlating the second detection signal to the presence or absence of C-reactive protein in the vaginal sample.

2. The method of claim 1, wherein the first detection signal is indicative of bacterial vaginosis.

3. The method of claim 1, wherein the second detection signal is indicative of premature membrane rupture.

4. The method of claim 1, wherein the amines include putrescine, cadaverine, tyramine, trimethylamine, or a combination thereof.

5. The method of claim 1, wherein the first detection zone contains an amine-sensitive chromogen capable of producing the first detection signal.

6. The method of claim 5, wherein the amine-sensitive chromogen includes phenol or a derivative thereof.

7. The method of claim 6, wherein the amine-sensitive chromogen further comprises an alkali metal chloride salt, oxyhalogenation catalyst, pH modifier, or a combination thereof.

8. The method of claim 1, wherein the lateral flow device is in fluid communication with detection probes conjugated with a specific binding member, the detecting probes being capable of producing the second detection signal when present in the second detection zone.

9. The method of claim 8, wherein the second detection zone contains an immunoreactive receptive material configured to preferentially bind with the specific binding member or C-reactive protein.

10. The method of claim 9, wherein the receptive material and specific binding member are antibodies of C-reactive protein.

11. The method of claim 1, wherein the lateral flow device includes a reagent zone located upstream from the first detection zone and the second detection zone.

12. The method of claim 1, wherein the reagent zone contains an electron donor containing halogen ions, oxyhalogenation catalyst, pH modifier, or a combination thereof.

13. The method of claim 1, wherein the reagent zone contains detection probes conjugated with a specific binding member.

14. The method of claim 1, wherein the chromatographic medium is a porous membrane.

15. A diagnostic test kit for identifying the onset of premature membrane rupture in a pregnant female, the kit comprising:
detection probes conjugated with a specific binding member; and
a lateral flow device that contains a chromatographic medium, the medium defining:
a first detection zone that contains an amine-sensitive chromogen capable of producing a first detection signal, the first detection signal indicating the presence or absence of amines in the vaginal sample; and
a second detection zone that contains an immunoreactive receptive material configured to preferentially bind with the specific binding member or C-reactive protein, wherein the detection probes are capable of producing a second detection signal when present in the second detection zone, the second detection signal indicating the presence or absence of C-reactive protein in the vaginal sample.

16. The diagnostic test kit of claim 15, wherein the amine-sensitive chromogen includes phenol or a derivative thereof.

17. The diagnostic test kit of claim 16, wherein the amine-sensitive chromogen further comprises an electron donor that contains halogen ions.

18. The diagnostic test kit of claim 17, wherein the electron donor is an alkali metal chloride salt.

19. The diagnostic test kit of claim 16, wherein the amine-sensitive further comprises an oxyhalogenation catalyst, pH modifier, or a combination thereof.

20. The diagnostic test kit of claim 15, wherein the lateral flow device includes a reagent zone located upstream from the first detection zone and the second detection zone.

21. The diagnostic test kit of claim 20, wherein the reagent zone contains an electron donor containing halogen ions, oxyhalogenation catalyst, pH modifier, or a combination thereof.

22. The diagnostic test kit of claim 20, wherein the reagent zone contains the detection probes.

23. The diagnostic test kit of claim 15, wherein the receptive material and specific binding member are antibodies of C-reactive protein.

24. The diagnostic test kit of claim 15, wherein the chromatographic medium is a porous membrane.

25. The diagnostic test kit of claim 15, wherein the lateral flow device further contains an absorbent material positioned downstream from the first detection zone and the second detection zone.
